(19) United States
(12) Patent Application Publication
(10) Pub. No.: US 2007/0154549 A1
(43) Pub. Date: Jul. 5, 2007

(54) Multiparticulate formulations for oral delivery

(75) Inventors: David Morton, Wiltshire (GB); David Simpson, Wiltshire (GB); John Staniforth, Wiltshire (GB)

Correspondence Address: DAVIDSON, DAVIDSON & KAPPEL, LLC, 485 SEVENTH AVENUE, 14TH FLOOR, NEW YORK, NY, 10018, US

(73) Assignee: VECTURA LTD, WILTSHIRE (GB)

(21) Appl. No.: 10/585,021

(22) PCT Filed: Dec. 24, 2004
(86) PCT No.: PCT/GB04/50047

§ 371 Date: Sep. 22, 2006

(30) Foreign Application Priority Data
Dec. 31, 2003 (GB) 2004/050047

(51) Int. Cl. A61K 9/26

(52) U.S. Cl. 424/470

(57) ABSTRACT

The present invention is directed to multiparticulate formulations for oral use, preferably comprising one or more therapeutically active agents. In particular, the present invention relates to fast melt formulations which are capable of masking the taste of the active agent, by virtue of one or more tastemasking measures, whilst retaining the desired drug dissolution profile and good mouthfeel. The multiparticulate formulations of the invention can be used in a multiple dose delivery device which dispenses a unit dose of the powder upon actuation, or can be packaged for dispensation in sachets or like unit dose containers.

The present invention is directed to multiparticulate formulations for oral use, preferably comprising one or more therapeutically active agents. In particular, the present invention relates to fast melt formulations which are capable of masking the taste of the active agent, by virtue of one or more tastemasking measures, whilst retaining the desired drug dissolution profile and good mouthfeel. The multiparticulate formulations of the invention can be used in a multiple dose delivery device which dispenses a unit dose of the powder upon actuation, or can be packaged for dispensation in sachets or like unit dose containers.

The most prominent mode of delivery of therapeutic agents is by the oral route, by means of solid dosage forms such as tablets and capsules. Oral administration of solid dosage forms is more convenient and accepted than other modes of administration, e.g., parenteral administration. However, the manufacture, dispensing and administration of solid dosage forms are not without associated problems and drawbacks.

With the manufacture of solid dosage forms, in addition to the active agent, it is necessary to combine other ingredients in the formulations for various reasons, such as to enhance physical appearance, to provide necessary bulk for tableting or capsuling, to improve stability, to improve compressibility or to aid in disintegration after administration. However, these added excipients have been shown to adversely influence the release, stability and bioavailability of the active ingredient.

The added excipients are a particular problem with drugs which require a high dose in order to provide a therapeutic effect, e.g., biphosphonate drugs. The inclusion of the additional excipient can make the final tablet extremely large which could result in esophageal damage due to the physical characteristics of the dosage form if it is not swallowed properly. Esophogeal damage can also be caused by toxicity caused by the drug itself, if the tablet becomes lodged in the throat or has an increased transit time through the esophagus, due to its increased size.

Further, the tableting of certain drugs has many associated production problems. In particular, many drugs, e.g., paracetamol (acetaminophen), have poor compressibility and cannot be directly compressed into solid dosage forms.

Consequently, such drugs must either be wet granulated or manufactured in a special grade in order to be tableted, which increases manufacturing steps and production costs.

The adherence to good manufacturing practices and process controls is essential in order to minimize dosage form to dosage form and batch to batch variations of the final product. Even strict adherence to these practices still is not a guarantee that acceptable variation will occur.

With the high cost of industrial scale production and governmental approval of solid dosage forms, such formulations are often available in a limited number of strengths, which only meet the needs of the largest sectors of the population.

Unfortunately, this practice leaves many patients without acceptable means of treatment and physicians in a quandary with respect to individualizing dosages to meet the clinical needs of their patients.

The dispensing of oral solid dosage forms also makes the formulations susceptible to degradation and contamination due to repackaging, improper storage and manual handling.

There are also many patients who are unable or unwilling to take conventional orally administered dosage forms. For some patients, the perception of unacceptable taste or mouthfeel of a dose of medicine leads to a gag reflex action that makes swallowing difficult or impossible. Other patients, e.g., pediatric and geriatric patients, find it difficult to ingest typical solid oral dosage forms, e.g., due to tablet size.

Other patients, particularly elderly patients, have conditions such as achlorhydria which hinders the successful use of oral solid dosage forms. Achlorhydria is a condition wherein there is an abnormal deficiency or absence of free hydrochloric acid in the gastric secretions of the stomach. This condition hinders the usual disintegration and/or dissolution of oral solid dosage forms, particularly dosage forms with high or insoluble excipient payloads. The present invention relates to fast melt multiparticulate dosage forms, which do not need to undergo disintegration and/or dissolution to the same extent as solid dosage forms. Therefore, this mode of administration is not affected by conditions such as achlorhydria.

Flavoured solutions/suspensions of some therapeutic agents have been developed to facilitate the oral administration of oral agents to patients normally having difficulty ingesting conventional solid oral dosage forms. While liquid formulations are more easily administered to the patient, liquid/suspension formulations are not without their own significant problems and restrictions. The liquid dose amount is not as easily controlled compared with tablet and capsule forms and many therapeutic agents are not sufficiently stable in solution/suspension form. Indeed, most suspension type formulations are typically reconstituted by the pharmacist and then have a limited shelf-life, even under refrigerated conditions. Another problem with liquid formulations, which is not as much a factor with tablets and capsules, is the taste of the active agent. The taste of some therapeutic agents is so unacceptable that liquid formulations are not a viable option. Further, solution/suspension type formulations are typically not acceptable where the active agent must be provided with a protective coating, e.g. a tastemasking coating or an enteric coating to protect the active agent from the strongly acidic conditions of the stomach.

Fast melt drug formulations have also been developed to facilitate the oral administration of oral agents to patients normally having difficulty ingesting conventional solid oral dosage forms. Fast melt formulations are typically in the form of tablets or lozenges that dissolve or disperse in a patient's mouth within a minute without the need of water or chewing. Drug delivery formulations which exhibit fast melt properties can improve patient compliance due to the ease of swallowing as well as the absence of a need for the co-administration of water or another fluid. Further, fast melt systems can be formulated as to have a superior taste and improved accuracy of dosing as compared to liquid preparations.

Other formulations which have been contemplated in order to facilitate the oral administration of oral agents and to avoid the associated problems of solid dosage forms are multiparticulate dosage forms as disclosed in WO 01/64182, the contents of which is hereby incorporated by reference.

Improvements in fast melt formulations were disclosed in WO 03/074029, the contents of which is also hereby incorporated by reference. This earlier patent application discloses a drug formulation for gastrointestinal deposition, the formulation comprising a free flowing plurality of particles comprising an active agent and a water soluble excipient, wherein the particles have a mean diameter of greater than about 10 μm to about 1 mm, and the formulation is capable of dissolving or dispersing in a patient's mouth within 1 minute after administration without the co-administration of a fluid. These fast melt formulations are said to exhibit the benefits of fast melt formulations as well as the benefits of multiparticulate formulations. It also said that the formulations facilitate the delivery of a wide range of therapeutic agents for gastrointestinal deposition and minimize pulmonary deposition of materials having undesirable or unknown pulmonary toxicology but which are approved for oral delivery.

WO 03/074029 also discloses a drug formulation for gastrointestinal deposition, the formulation comprising a free flowing plurality of particles and including an active agent and a water soluble excipient, wherein the particles have a mean diameter of greater than about 10 μm to about 1 mm, and the excipient has a negative heat of solution. These formulations have the advantage that, when administered via the oral cavity, the local cooling caused by the water soluble excipient dissolving in saliva serves to mask the taste of the active agent in a manner which does not delay the release, or dissolution of the active agent itself. Preferably, these formulations are capable of dissolving or dispersing in a patient's mouth within one minute after administration, without the co-administration of a fluid.

The fast melt formulations disclosed in this earlier application may include particles which each include both active agent and water soluble excipient. The particles can comprise a core and a coating, with the coating including a quantity of the water soluble excipient. For low-dose embodiments, the coat could also contain the active.

According to a method of preparing fast melt formulations disclosed in WO 03/074029, the particles are formed by melt-coating core particles with a coating material that includes (and may consist of) a quantity of the excipient, at a temperature below that at which the active agent melts or decomposes. Forming the particles in this manner is considered to provide them with surface properties that tender them easily wetted and capable of rapidly absorbing water from their environment and, thus, able to facilitate the rapid dissolution or dispersion of the formulation, especially the active agent, when the formulation is exposed to an aqueous environment, such as in the oral cavity.

The method involves forming particles by melt-coating core particles with a coating material that includes a quantity of the water soluble excipient, at a temperature below the melting point or decomposition temperature of the active agent.

Whilst the fast melt formulations known from the prior art have a number of good properties and tend to release the active agent well, it has been found that the masking of the taste of the active agent can be poor and this is a problem, especially where the active agent has a particularly unpleasant taste, as is the case with paracetamol and ibuprofen, for example.

In the past, attempts to mask the taste of active agents in fast melt formulations have either failed to mask both the initial taste of the active agent and its aftertaste, or have adversely affected the dissolution profile of the active agent, so that the onset of the therapeutic effect is delayed.

One approach to tastemasking in the prior art is to include flavouring agents in the formulations. However, whilst these agents tend to provide an initial burst of flavour which covers the initial taste of the active agent, this masking is relatively short-lived and it is not able to mask the often unpleasant aftertaste adequately.

Attempts to mask the taste of the active agent, including the aftertaste, have included coating the active particles with a material which prevents the active particles from dissolving in the mouth. If the particles do not dissolve, there will be no taste of the active agent to be detected by the subject. This approach has the disadvantage that it gives the fast melt formulations a gritty mouthfeel when administered, as a result of the active particles which do not dissolve. A further disadvantage associated with this approach is that it necessarily changes the dissolution profile of the formulation, delaying the release of the active agent from the formulation and thereby delaying the onset of the therapeutic effect.

It is therefore an aim of the present invention to provide fast melt formulations which combine good tastemasking and retention of the desired rapid dissolution profile, together with the provision of a pleasant mouthfeel.

The applicants have discovered that this aim may be achieved by one or more of a number of measures, each of which help to conceal the taste of the active agent included in the fast melt formulation without slowing the release of the active agent and whilst retaining good mouthfeel with no feeling of grittiness.

In a first aspect of the present invention, an improved fast melt formulation comprising a free-flowing plurality of particles is provided, comprising a pharmaceutically active agent and an excipient, wherein the formulation includes tastemasking agents which are capable of substantially masking the initial taste of the active agent and its aftertaste, whilst having substantially no effect on the dissolution of the formulation compared to a formulation without the tastemasking agents.

In one embodiment of the present invention, the effect of the tastemasking agents included in the formulation is enhanced by ensuring that the active agent is completely covered by a fast melt coating. The formulation may comprise core particles made up of the pharmaceutically active agent, and/or excipients. These core particles are then coated, preferably using a melt coating method, with a mixture comprising a melt binder material and an excipient, which includes tastemasking agents.

Methods for forming the melt coating on the core particles are known in the prior art. However, it has been surprisingly discovered that the coating of the core particles with excipients and melt binder disclosed in the prior art is not always completely effective. It is clearly crucial to ensure that the core particles are completely and uniformly coated with a mixture of excipients and melt binder. If the coating is a partial or discontinuous coating, the active agent is effectively exposed upon administration of the formulation and the taste of the active agent will be difficult, if not impossible, to mask.

It is also essential that each core particle is surrounded by a coating within which the excipient particles are evenly distributed. If this does not happen, the tastemasking of the active agent present in the core particles may be compromised.

In order to ensure that the melt coating is predictable and effective, it has been surprisingly found that the particle sizes of the excipient and the core particles play a significant part in the coating process and determine, to an extent, the nature of the coating achieved. Firstly, it has been found that the smaller the particle size of the excipient, the more effectively it will be dispersed on the surface of the core particles during the melt coating process. This results in more excipient being incorporated into the coating and better distribution of the excipient within the coating. Secondly, it has been found that, if the particle size distribution of the excipient or excipients is similar to that of the core particles, the excipient particles tend to agglomerate instead of being dispersed on the surface of the core particles. This is to be avoided, in order to ensure that as much of the excipient as possible coats the core particles. Agglomeration of the excipient particles (sometimes with particles of the melt binder) will result in discontinuous drug-particle coating and inefficient tastemasking.

Thus, in a preferred embodiment of the present invention, the preparation of melt-coated particles according to the present invention involves a step wherein the size of the particles of the various component materials is controlled or selected. In some embodiments, the melt coating process will result in a continuous coating around each of the core particles.

Prior art methods of melt coating have not involved any control of the size of the excipient particles or of the particle size distribution of the excipient particles and the drug particles. Indeed, until now, it would appear that the significance of these factors in the melt coating process has been completely overlooked.

However, it has now been realised that these factors determine the effectiveness of the coating, in particular in relation to the masking of the taste of the active agent in the core particles.

In some embodiments of the present invention, the core particles have a particle size of between 10 and 1000 μm. Preferably, the core particles have a particle size of between 100 and 300 μm, or between 200 and 600 μm.

Ideally, the particle size of the excipient should be less than that of the core particle. In one embodiment, the excipient particle size is approximately 10% or less of the size of the core particle size. Despite the foregoing, it should be noted that formulations have been produced where the particle size of the excipients has not been controlled yet the formulation was organoleptically acceptable.

In an ideal formulation according to the present invention, all of the excipient material would be incorporated into the coating. The more excipient that is incorporated in the coating, the more efficient the tastemasking. As mentioned above, controlling the particle size of the excipients should improve the efficiency of the coating process, assisting the incorporation of the excipient material into the coating.

The thickness of the coating will be dependent upon the nature and the amount of melt binder. The thickness of the coating, and the nature and the amount of the melt binder is thought to influence both tastemasking and dissolution.

The effective coating described above is particularly significant where each of the core particles includes the active agent. This will be the case where the drug payload is relatively high, for example where the formulation comprises an active agent such as, for example, paracetamol, clarithromycin and valproic acid.

Furthermore, this effective coating is also particularly significant where there is no pre-coating of the core particles in order to provide additional tastemasking or in order to modify release of the drug.

A quantity of the active agent can be included in the core or core particles and/or in the coating or coating material. In some preferred embodiments, the coating or coating material is substantially free of active agent, whereas in others, the core is substantially free of active agent.

The coating or coating material may comprise a water soluble, hydrophobic or hydrophilic binder. Preferably, the binder melts or softens sufficiently to melt-coat the core particles at a temperature below that at which the active agent melts or decomposes. Furthermore, the water soluble excipient preferably melts or softens sufficiently to melt-coat the core particles at a temperature below that at which the active agent melts or decomposes. In further preferred arrangements, the binder melts or softens sufficiently to melt-coat the core particles at a temperature below that at which the water soluble excipient melts or decomposes.

In some embodiments, the coating or coating material substantially completely covers the surface of the core or core particles. Thus, the formulation can comprise a core that consists substantially or entirely of active agent surrounded by a coating that comprises water soluble excipient either alone, or in combination with a water-soluble or hydrophilic binder. When the water soluble excipient is employed alone in such particles, it is preferred for it to be capable of melting or softening sufficiently to melt-coat the core particles at a temperature below that at which the active agent melts or decomposes. Where a binder is employed, the water soluble excipient need not be capable of melting or softening at a temperature below the melting or decomposition temperature of the active agent. However, when such a high melting point water soluble excipient is employed, the binder should be capable both of melting or softening sufficiently to melt-coat the core particles at a temperature below that at which the active agent melts or decomposes, and of binding the water soluble excipient in the coating.

Suitable materials for use as melt binders in the present invention have a melting point in the range of 40° C. to 150° C. In some embodiments, the melt binders are water soluble melt binders such as sugar alcohols, for example xylitol and erythritol, or polyethylene glycols (PEGs) such as PEG4000 and PEG6000, PEG8000, PEG120000 and PEG20000, as well as poloxamers.

Some of the above discussed components of the formulations according to the present invention can only be incorporated into the formulations when they are prepared in the absence of water. If these components were to be exposed to water, they would dissolve and could not be incorporated in the formulation in the form of the described melt coating. Examples of such water-sensitive components include sodium starch glycolate and the effervescent component, which must be incorporated in the absence of water for the optional effect upon administration of the dosage form.

It is also possible to use hydrophobic melt binders in the formulations of the present invention. Examples of suitable hydrophobic melt binders include stearic acid, glyceryl palmitostearate and glyceryl monostearate. Some of the waxes which are used in suppository bases and which are licensed for use in oral formulations may also be employed in the formulations of the present invention. Such hydrophobic melt binders will obviously not dissolve as rapidly as the water soluble binders upon administration. However, these hydrophobic binders may be included in the formulation in quantities without having significant effect on the release of the active agent. The effect of a hydrophobic melt binder on the release of the active agent is expected to depend, to a certain extent, on the nature of the drug. There is evidence that, for a freely soluble drug such as chlorpheniramine maleate, levels of hydrophobic melt binder of up to 20% do not delay drug release.

In an alternative embodiment, such hydrophobic melt binders could be selected to also have a release-modifying effect. In such circumstances, the hydrophobic binder is present in a higher concentration, so that it does affect dissolution of the particles and release of the active agent.

The inclusion of hydrophobic components in the formulations of the present invention has the added advantage that it may reduce, or even prevent, the ingress of water into the formulation whilst it is being stored. Clearly, the formulations and in particular the water soluble excipients, are water-sensitive and preventing the ingress of water could significantly increase the shelf-life of these formulations.

The core or core particles, in addition to including active agent, can also include a quantity of the water soluble excipient and/or an additional excipient, which may also be water soluble, but which does not necessarily qualify as a water soluble excipient in accordance with the present invention. For example, the core can comprise a granulation of such an additional excipient (e.g. polyvinyl alcohol, or polyvinylpyrrolidine) and active agent, or can comprise a particle (e.g. a microcrystalline cellulose sphere) of additional excipient coated with active agent.

In other embodiments, the core can consist entirely of water soluble excipient. In such embodiments, the coat or coating material comprises active agent and either an additional quantity of water soluble excipient, or a binder. When the coat or coating material comprises active agent and binder, additional water soluble excipient can also be present in therein.

The water soluble excipient is preferably a sugar, sugar alcohol, polyethylene glycol (PEG), or polyethylene oxide, and is preferably not lactose. Formulations in accordance with the invention, preferably, are lactose free. The preferred water soluble excipients are the sugar alcohols including, but not limited to sorbitol, mannitol, maltitol, reduced starch saccharide, xylitol, reduced paratinose, erythritol, and combinations thereof. The preferred sugar is glucose. Other suitable water-soluble excipients include gelatin, partially hydrolyzed gelatin, hydrolyzed dextran, dextrin, alginate and mixtures thereof. Also suitable as water soluble excipients are sodium bicarbonate, citric acid, tartaric acid, malic acid, fumaric acid, adipic acid, succinic acid and sodium glycine carbonate. Other water soluble excipients will also include the water soluble components discussed above, such as the sweeteners and the effervescent components.

The melting point of the melt binder incorporated in the coating is preferably equal to or below 150, 120 or 110° C., and is preferably at least 40 or 50° C. Preferably, the excipient melts at around or below 100° C. In certain embodiments, any excipient included in the core particles has a melting point higher than that of the melt binder.

In certain embodiments of the present invention, the melt binder can be a water soluble excipient.

In one embodiment of the present invention, the water soluble excipient has a heat of solution equal to or below −7 kcal/kg. More preferably, the heat of solution of the water soluble excipient is equal to or below −10, −15, −20, −25, or −30 kcal/kg. Sugar alcohols are examples of water soluble excipients with a negative heat of solution.

In another embodiment, the solubility in water of the water soluble excipient is preferably at least 20, 30 or 40% w/w at 25° C.

It is preferred that formulations are formed by a process in which the active agent is not raised to or above its melting point, above its softening point or above a temperature at which a significant proportion thereof is caused to decompose.

The core particles of the fast melt formulations according to the present invention may be pre-coated, prior to the melt-coating process. The pre-coating may be to assist tastemasking where the active agent has an unpleasant taste which is particularly difficult to mask, or where the release of the active agent from the core particles is to be modified, for example where a sustained release profile is desired.

In further embodiments of the present invention, the core or core particles include an additional excipient for controlling or delaying the release of the active agent. In this regard, the core or core particles can include a layer or coating of such an additional excipient encapsulating an inner core comprising the active agent. The additional excipient can be selected from those known to persons skilled in the art to be capable of controlling the release of an encapsulated active agent. Such excipients include those commonly used to provide enteric and sustained release coatings.

Examples of the former include cellulose acetate phthalate, hydroxypropyl-methylcellulose phthalate, polymethacrylates, such as Eudragit® L 100-55 or L 30 D-55, and shellac. Examples of the latter include ethylcellulose, hydroxypropyl-cellulose, hydroxypropylmethylcellulose, and polymethacrylates, such as Eudragit® RL and RS film-coating systems.

In alternative embodiments, formulations can provide rapid release of the active agent. In this regard, the term “rapid release” should be understood to mean that such formulations release at least 80% of their active agent within 45 minutes in standard dissolution tests. In the case of poorly soluble active agents, such formulations typically release at least 80% of their active agent within 40, 30, 20, 15 and preferably 10 minutes after being administered to a patient's oral cavity. In the case of more soluble active agents, such formulations typically release at least 80% of their active agent within 10, 7 and preferably 5 minutes after being administered to a patient's oral cavity. In particularly preferred embodiments of the invention, the active agent will dissolve into an aqueous environment more rapidly from a formulation in accordance with the invention than it would if it had not been incorporated in such a formulation.

The dissolution or dispersion of the formulation can be improved with the use of a surfactant, such as sodium lauryl sulphate (Texapon K 12), various polysorbates known under the trade name Tween, ethers of polyhydroxy ethylene fatty acids known under the trade name Brij, esters of polyhydroxy ethylene fatty acids known under the trade name Myrj, sodium desoxycholate, glycerol polyethylene glycol ricinoleate (Cremophor EL), polyoxyethylene-polyoxyptopylene polymers known under the trade name Pluronic, and various polyalkoxy alkylene sterol ethers.

The fast melt formulations of the present invention can also comprise starches, e.g., corn starch, or modified starches, e.g., sodium starch glycolate or mixtures thereof, in any proportions. Starches can provide increased salivation due to the porous nature of the starch. Increased salivation favours rapid dissolution or dispersion of the formulation upon oral administration.

When a starch is present in the formulation, the formulation can further comprise a starch degrading enzyme will have a synergistic effect with the starch with respect to dissolution or dispersion. The enzymes upon being contacted with an aqueous solution will initiate conversion of the starch to mono and polysaccharides which quickly dissolve in the aqueous environment and further contribute to improving the taste of the multiparticulate formulation and increasing salivation.

The enzymes can be chosen for their degradation effect on the starch and also for their stability over time, i.e. during the shelf-life of the fast melt multiparticulate formulation. Advantageously, the enzyme will be chosen from the group of starch degrading enzymes comprising alpha-amylase, beta-amylase, amyloglucosidase, debranching enzymes and glucose-fructose isomerase. In certain embodiments, the enzymes can be an equal mixture of amyloglucosidase and a-amylase.

In certain embodiments, drug formulations in accordance with the invention are prepared by a process comprising melt granulating the water soluble excipient and the active agent to form a homogenous mixture. In an alternate embodiment, the process comprises melt coating the water soluble excipient onto the active agent which can be optionally pregranulated with a pharmaceutically acceptable excipient.

In such processes, the water soluble excipient is preferably a water soluble alcohol such as xylitol.

The melt granulation and melt coating processes are particularly preferred processes of the present invention as it is not necessary to use an aqueous fluid as a processing aid. This results in a process which can be used for a wide variety of active agents, including those agents which would be susceptible to degradation upon contact with water. Accordingly, such processes provide advantages over many prior art processes for making fast melt systems which rely on water as a processing aid. These prior art processes would not be suitable for water labile drugs as such processes would result in degradation of the drug during the manufacturing process and during storage due to residual moisture in the final product.

In certain embodiments, formulations in accordance with the invention can be prepared by subliming solvent from a composition comprising the active agent and the water soluble excipient and reducing the sublimed composition to the particles.

In such embodiments, the composition can further comprise an excipient selected from the group consisting of polyvinyl alcohol, polyvinylpyrrolidone, acacia or a combination thereof. The sublimation is preferably by freeze-drying and the solvent can be an aqueous solvent or a co-solvent comprising an aqueous solvent and an alcohol. A surfactant can also be included in such a formulation.

In certain embodiments, fast melt formulations in accordance with the invention can be prepared by a process which comprises preparing a mixture comprising the active agent, the water soluble excipient and a solvent, freezing the mixture, vacuum drying the frozen mixture above a collapse temperature of the mixture to form a partially collapsed matrix network and reducing the sublimed composition to the particles. Preferably, the mixture comprises the active agent, a gum, a carbohydrate base, and a solvent, wherein the gum is selected from the group consisting of acacia, guar, xanthan, tragacanth gum, and mixtures thereof, and the carbohydrate is selected from the group consisting of mannitol, dextrose, sucrose, lactose, maltose, maltodextrin, corn syrup solids, and mixtures thereof.

In certain embodiments, fast melt formulations in accordance with the invention can be prepared by a process which comprises preparing a mixture comprising the active agent, the water soluble excipient and an agar aqueous solution, solidifying the mixture into a jelly form, drying the jelly and reducing the dried composition into the particles. The drying can be effected by reduced pressure drying, aeration drying or freeze-drying.

In certain embodiments, fast melt formulations in accordance with the invention can be prepared by a process which comprises melt spinning the active agent with the saccharide to form a mass of spun fibres and reducing the spun fibres to the particles. The saccharide can be sucrose or glucose.

In order to achieve the desired lower limit of the particle size of the fast melt multiparticulate formulation of the invention, air jet sieving can be used to remove fine particles. In particular embodiments, the invention is directed to a method of preparing a multiparticulate drug formulation for gastrointestinal deposition comprising preparing a non-compressed free flowing plurality of particles comprising a core comprising a drug and a pharmaceutically acceptable excipient as disclosed herein and air jet sieving the particles to separate the cores from fine particles; and thereafter overcoating the core with a functional coating as disclosed herein.

The present invention is also directed to compositions obtained using these methods.

For purposes of the present invention, the term “device” refers to an apparatus capable of delivering a unit dose of drug.

The term “system” refers to a drug delivery device in combination with a fast melt multiparticulate formulation having the specifications disclosed herein, e.g. drug particle size, excipient type, etc.

The term “discreet collection” refers to a non-compressed free flowing unit of multiparticulates with minimal particulate matter being dispersed in the surrounding environment (e.g., as a cloud or mist).

The term “drug” refers to any agent which is capable of providing a therapeutic effect to a patient upon gastrointestinal deposition. This encompasses all drugs which are intended for absorption for a systemic effect (regardless of their actual bioavailability) as well as drugs intended for a local effect in the gut and/or oral cavity, e.g. nystatin, antibiotics or local anesthetics.

The term “particle size” refers to the diameter of the particle.

The term “deposition” means the deposit of the unit dose at the intended point of absorption and/or action. For example, gastro-intestinal deposition means the intended deposit of the unit dose in the gastrointestinal system for e.g., absorption for a systemic effect or to exert a local effect Pulmonary deposition means the intended deposit of drug into the lungs in order to provide a pharmaceutical effect, regardless that the unit dose may enter the oral cavity prior to pulmonary deposition.

The term “dispense”, when used in connection with the devices and systems of the present invention, means that the device or system delivers the unit dose ex viva with the intent of subsequent administration to a mammal. For example, the device or system can dispense the unit dose into a food, a liquid, a spoon, or another intermediate receptacle.

The term “administer”, when used in connection with the devices and systems of the present invention, means that the device or system delivers the unit dose in vivo, i.e., directly into the gastrointestinal tract of a mammal.

The term “deliver” is meant to cover all ex vivo and in vivo delivery, i.e., dispensing and administering, respectively.

The term “patient” refers to humans as well as other mammals in need of a therapeutic agent, e.g., household pets or livestock This term also refers to humans or mammals in need of or receiving prophylactic treatment.

The term “fast melt” means a formulation which dissolves or disperses in a patient's mouth within 1 minute after administration without the co-administration of a fluid. Preferably, the formulation dissolving or disperses in a patient's mouth within 30 seconds, or 15 seconds after administration without the co-administration of a fluid.

The term “disperses” means that the administered formulation becomes hydrated in the mouth and the particles of the formulation become suspended is saliva, such that the multiparticulate formulation is wetted and easily swallowed.

In certain embodiments, the particulates are of a size such that an effective dose cannot be delivered into the lower lung of a human patient. This should be understood to mean that a small percentage of drug (but not an amount effective to render a therapeutic effect) may in fact be inadvertently delivered to the lungs of the patient.

Also, the present invention is not limited to the treatment of humans alone. The invention ray be used for delivering doses of drugs to other mammals as well.

In this specification, there are references to the temperature at which the active agent or the water soluble excipient decomposes. This temperature should be understood to be the temperature at and above which the active agent or excipient would decompose to a significant extent, if held there for sufficient time for the active agent or excipient to be processes by melt granulation.

As briefly discussed above, the previous efforts to mask the taste of the active agent in fast melt formulations has generally failed to completely mask the taste. This is partly due to the flavour profiles of the formulations. The fast melt formulations are generally designed to allow fast dissolution of the formulation, resulting in release of the active agent. Upon contact with the saliva of the patient's mouth, the formulation immediately begins to dissolve and the particulate formulation dissolves to form a syrup-like dispersion which can be comfortably swallowed.

Unless the core particles are coated with a coating which delays any release of the active agent until after the formulation has been swallowed, the active agent will be available for detection by the patient's taste receptors. Following a brief period directly following the administration of the formulation during which the active agent is still coated, the taste intensity of the active agent will quickly peak and, within a matter of seconds, the intensity will drop. However, following the peak of active agent taste intensity, the taste does not disappear. Rather, the taste intensity plateaus and it actually maintained at a reduced level for a relatively long period of time. This is the “aftertaste”.

In contrast, the flavouring agents simply added to fast melt formulations in the past have exhibited a flavour intensity peak which exceeds that of the active agent. However, the flavour intensity of the flavouring agents drops off more rapidly than that of the active agent, so that the intensity of flavouring drops below that of the active agent shortly after the peak, and the masking fails so that the patient can taste the active agent.

Coating the core particles so that the release of the active agent is retarded can result in a reduction in the intensity of the flavour of the active agent, so that the intensity of the flavouring agents exceeds that of the active agent throughout, and the patient cannot taste the active agent. However, as previously explained, this approach has an adverse effect on the release of the active agent and results in an unpleasant, gritty mouthfeel.

It is therefore a further aim of the present invention to provide a formulation wherein the flavouring intensity substantially always exceeds the intensity of the taste of the active agent, without affecting the dissolution profile of the formulation. In particular, the tastemasking does not result in a significant slowing of the dissolution.

This aim is achieved by employing one or more of the following tastemasking means. It will be clear that these tastemasking means have been designed to be compatible with the preferred methods for preparing the fast melt formulations of the present invention.

Firstly, the tastemasking of a fast melt formulation may be enhanced by the addition of low viscosity polymers to the formulation. These polymers have been found to modify the mouthfeel and the aftertaste of the fast melt formulations. The low viscosity polymers appear to reduce the drug aftertaste by forming a physical barrier between the taste receptors of the mouth and the drug moiety. In order to elicit the effect on aftertaste, these low viscosity polymers should be incorporated into the coating.

Low viscosity maltodextrins may be used in this way to modify mouthfeel and mask any aftertaste of the active agent. Above certain concentrations, the inclusion of maltodextrin may retard drug release, and may therefore also be included as a drug release modifying agent. That said, the maltodextrin will usually be used in concentrations which will not affect release of the drug.

Another example of a suitable low viscosity polymer is low viscosity grade sodium starch glycolate, which exhibits the same effect on the mouthfeel and aftertaste as maltodextrin, but has been shown to disperse much more quickly. Sodium starch glycolate is used as a super-disintegrant and it is known to incorporate it in tablets formed by direct compression. However, sodium starch glycolate has not previously been included in fast melt formulations and its tastemasking and mouthfeel properties have not previously been recognised. It has also been found that the inclusion of high concentrations of sodium starch glycolate in a fast melt formulation comprising ibuprofen actually reduces the highly undesirable “afterburn” of this active agent, although this may be at the expense of mouthfeel. However, this loss of mouthfeel is acceptable, if the unpleasant taste of the ibuprofen can be masked effectively.

According to some embodiments of the present invention, sodium starch glycolate may be included in the formulations in the non-active core, to aid dispersion of the formulation upon administration.

Other low viscosity polymers which may be included in fast melt formulations in order to enhance tastemasking and mouthfeel include alginates and xanthan.

Further tastemasking of the active agent can be achieved by including an effervescent agent in the fast melt formulation. Where the formulation includes a soluble acid source and an alkali metal carbonate or carbonate source, these interact upon dissolution (that is, upon administration of the formulation) to produce carbon dioxide. The carbon dioxide has been found to aid dispersion of the formulation and it also improves mouthfeel. What is more, the carbon dioxide may also contribute to tastemasking the active agent.

A variety of materials may be used as the effervescent material forming carbon dioxide. The carbonate sources can be selected from the group consisting of dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate and amorphous calcium carbonate. The weak acids may include, for example, citric acid, tartaric acid, malic acid, fumaric acid, adipic acid, succinic acid, acid anhydrides thereof, acid salts thereof and combinations thereof.

In addition to their role in the generation of carbon dioxide, the weak acids also act as salivary stimulants. The increased levels of saliva will assist with the dissolution and/or dispersion of the multiparticulate formulation.

The traditional methods for masking unpleasant tastes involve the use of flavouring agents and sweeteners. These may also be included in the formulations according to the present invention.

In some embodiments of the invention, the formulations include one or more sweeteners, such as water soluble artificial sweeteners, including but not limited to soluble saccharin salts, such as sodium or calcium saccharin salts, cyclamate salts, acesulfame-K, the free acid form of saccharin and mixtures thereof. The sweetener can also comprise a dipeptide based sweetener such as L-aspartyl L-phenylalanine methyl ester. The use of sweeteners needs to balance the intensity of the sweetness with its ability to mask any unpleasant taste from the active agent.

Particularly preferred sweeteners are acesulfame potassium (also referred to herein as acesulfame K), aspartame, suctalose and sodium saccharin, and combinations thereof. In a particular embodiment of the invention, a mixture of acesulfame potassium and aspartame is used, and a 50:50 ratio of these sweeteners has been found to be particularly effective.

Sugar alcohols or polyols may also be used as sweeteners in the formulations according to the present invention. Polyols, like non-nutritive sweeteners, are non-cariogenic, and used frequently in “sugar-free” products. Some sugar alcohols also have a negative heat of solution and this is an attractive property in the formulations of the invention. The cooling effect these sweeteners have is thought to further reduce the perception of the taste of the active agent.

Examples of polyols which may be used in the formulations of the present invention include xylitol, sorbitol mannitol and maltitol. The degree of cooling depends on various criteria, such as heat of solution, solubility and particle size. The finer the particle, the more quickly it dissolves into solution, and therefore, the greater the cooling sensation. Erythritol has the greatest negative heat of solution and xylitol the next greatest.

The water soluble excipient of the formulation can be a sugar alcohol including, but not limited to sorbitol, mannitol, maltitol, reduced starch saccharide, xylitol, reduced paratinose, erythritol, and combination thereof. Other suitable water-soluble excipients include gelatin, partially hydrolyzed gelatin, hydrolyzed dextran, dextrin, alginate and mixtures thereof.

According to another embodiment of the present invention, flavouring agents are included in the fast melt formulations, to make the product more palatable and to mask any unpleasant taste from the active agent.

A range of flavouring agents may be used in the formulation. These agents may be included by different means. For example, the flavouring agent may be present as fine spray-dried material or as larger encapsulated flavouring particles. The flavouring agents may be added to the formulation along with all of the other excipients at the melt-coating stage. However, due to the volatile nature of the flavouring materials, a change in the flavour profile has been detected upon prolonged exposure to elevated temperatures.

In order to avoid any change in the flavouring, in one embodiment of the invention, spray dried flavouring agents are added to the formulation only once it has almost completely cooled, but preferably whilst it is still “tacky”.

Alternatively, the spray dried flavouring materials may be simply dry blended with the cooled formulation. Where such dry blending is used, there is a risk that some segregation of the flavouring particles from the formulation may occur. This will clearly lead to variability in flavouring and tastemasking.

Where the flavouring agents are encapsulated, these larger particles may also be dry-blended with the formulation. Because of the larger size of these flavouring particles compared to the spray dried particles, segregation is not as likely, the encapsulated flavouring particles being approximately the same size as the active drug cores.

In a further aspect, the invention provides the use of a drug formulation in accordance with the first or second aspect of the invention, or a drug formulation prepared by a method in accordance with the third aspect of the invention, for the preparation of a medicament for treating a human or animal patient, wherein the formulation is administered directly and in an un-encapsulated form to the patient's oral cavity. The invention also provides a method of treating a human or animal patient, wherein a formulation in accordance with the first or second aspect of the invention, or prepared by a method in accordance with a third aspect of the invention, is administered in an un-encapsulated form directly into the patient's oral cavity.

It is also possible for formulations in accordance with either the first aspect or the second aspect of the invention to include additional particles with different properties to those described above. For example, the additional particles may not include any active agent.

Fast melt multiparticulate formulations in accordance with the invention are, preferably, divisible into unit doses (e.g. with the use of a multiple unit dosing device) with a weight uniformity which is within the acceptable range of weight uniformity for tablets or capsules. A detailed discussion of weight uniformity can be found in the USP/NF 23/18 section 905, which is hereby incorporated by reference in its entirety for all purposes.

The invention also provides methods of preparing fast melt multiparticulate dosage forms and systems disclosed herein. The invention further provides methods of preparing fast melt multiparticulate dosage forms without the use of an aqueous fluid as a processing aid.

The invention additionally provides methods of preparing multiple unit delivery systems containing fast melt multiparticulate dosage forms in accordance with the invention.

The invention further provides methods of administering an active agent comprising administering a fast melt multiparticulate dosage form.

The invention additionally provides methods of administering an active agent comprising administering a fast melt multiparticulate dosage form via the use of a multiple unit delivery system.

In certain embodiments, the present invention is directed to a drug formulation for gastrointestinal deposition comprising a non-compressed free flowing plurality of particles comprising an active agent and a water soluble excipient, the particles having a mean diameter of greater than 10 μm to about 1 mm, the particles comprising at least about 50% drug and the formulation dissolving in a patient's mouth within 1 minute after administration without the co-administration of a fluid.

In certain embodiments, the invention is directed to a method of treating a patient with an active agent for gastrointestinal deposition comprising administering a formulation comprising a non-compressed free flowing plurality of particles comprising an active agent and a water soluble excipient, the particles having a mean diameter of greater than 10 μm to about 1 mm, and the formulation dissolving in a patient's mouth within 1 minute after administration without the co-administration of a fluid.

In certain embodiments, the invention is directed to a drug delivery system for delivery of a drug for gastrointestinal deposition. The system comprises a multiple unit dosing device comprising a housing and an actuator, the device containing multiple doses of a fast melt multiparticulate formulation, the device upon actuation delivering a unit dose of the fast melt multiparticulates for gastrointestinal deposition, the multiparticulates having a mean particle size of greater than 10 μm and preferably less than about 1 mm in order to minimize pulmonary deposition of the multiparticulates and such that an effective dose of the drug cannot be delivered into the lower lung of a human patient. The drug delivery system can be used to administer the unit dose of fast melt multiparticulates into the oral cavity of the patient (int-vivo) or to dispense the unit dose into an intermediate receptacle (ex-vivo) for subsequent gastrointestinal deposition. Oral drug delivery systems and devices for oral powders are disclosed in WO01/64182, hereby incorporated by reference in its entirety for all purposes.

In certain embodiments, the invention provides a method of treating a patient in need of multiple doses of a drug for gastrointestinal deposition comprising preparing fast melt multiparticulates in a manner wherein the drug particles when placed in the oral cavity are not deposited in any substantial amount to the lungs and dissolve or disperse in the mouth within 1 minute after administration, placing multiple unit doses of the fast melt multiparticulates in a device which meters a single unit dose for delivery; and either (a) administering the unit dose into the oral cavity of a patient or (b) dispensing the unit dose into an intermediate receptacle and thereafter administering the unit dose into the oral cavity of the patient.

In certain embodiments, the particles of the invention comprise at least about 50% drug; at least about 60% drug; at least about 70% drug; at least about 80% drug; or at least about 90% drug. In others, low doses of up to 50%, 20%, 10% or 5% of drug or active agent are carried by the inventive particles. In certain embodiments, the invention provides a method for delivery of a drug comprising delivering fast melt multiparticulates comprising drug particles via the use of a multiple unit dosing device comprising a housing and an actuator, the device upon actuation delivering a unit dose of the fast melt multiparticulates, and thereafter re-using the device to deliver additional unit doses of the fast melt multiparticulates at appropriate dosing intervals.

In preferred embodiments of the invention, the unit dose comprises a discreet collection of fast melt multiparticulates. For purposes of the invention, a “discreet collection” means that the fast melt multiparticulates are in the form of a non-compressed free flowing unit and not dispersed in a cloud or mist, which effectively minimizes inhalation of the active agent into the lungs of the patient. The unit dose can include from about 0.01 mg to about 1.5 g of active agent. For example, the dose of active agent can be from about 1 mg to about 100 mg, or from about 10 mg to about 50 mg. Naturally, the formulations of the present invention may include combinations of two or more active agents. For example, a combination of paracetamol and phenylephrine may be included in the formulations.

In certain embodiments of the invention, the mean diameter of the fast melt multiparticulates is of a size which minimizes their capacity to be inhaled into the lower lung. Typically, the mean particle size of the drug particles (or agglomerates) is greater than 10 μm, preferably greater than about 50 μm or greater than about 75 μm. In certain embodiments of the invention, the mean particle size range of the drug particles is from about 100 μm to about 1 mm, preferably from about 50 μm to about 500 μm. In preferred embodiments, greater than 80% of the particles have the above disclosed diameter (not mean diameter), e.g. 80% of the drug particles have a diameter of greater than 10 μm, or a diameter of from about 100 μm to about 1 mm. In other embodiments, greater than about 90% of the particles have the above disclosed diameter.

In certain embodiments of the invention, the mean diameter of the fast melt multiparticulates does not vary by greater than about 20%, preferably not greater than about 15% and most preferably not greater than about 10%.

In certain embodiments of the invention, the multiple doses of the fast melt formulation are contained in a reservoir. The reservoir can contain an amount of multiparticulates to provide any number of unit doses, e.g. from about 2 doses to about 400 doses. For ease in patient compliance, the reservoir has a sufficient quantity of to provide e.g. a days supply, a months supply or a years supply of doses, e.g. 30 or 365 for once daily dosing for a month or year, respectively.

In order to aid in patient compliance, certain embodiments of the invention include a counter or indicator to display the number of doses remaining in the system or the number of doses actuated.

In certain embodiments of the invention, the unit doses are individually metered prior to actuation, e.g., in the form of capsules or blisters or preferably in the form of sachets, wherein each sachet contains one individual unit dose. The system can be capable of containing any multiple of pre-metered unit doses, e.g. from about 2 to about 400 sachets.

In general, it has been recognized in the art that dry powder inhalation or insufflation formulations must consist of particles of a size of about 2 μm in diameter in order for the particles, when inhaled, to reach the peripheral or “deep” lung, including alveoli. Particles larger than 10 μm in diameter are not able to reach the deep lung when inhaled because they are collected on the back of the throat and upper airways in humans. Therefore, known powder delivery systems have been formulated with particle sizes of less than 10 μm in order for the particles to reach the intended site of action, the pulmonary system.

As the fast melt multiparticulates of the present invention are not intended to be compressed, a high load formulation of the active agent is ascertainable. This is due to the fact that excipients which must be included in prior art fast melt tablets (e.g., fillers in order to provide bulk for tableting and disintegrants to provide a breakdown of the tablet upon administration) need not be included in the present formulations, or may be included to a lesser extent. As the fast melt formulations can have lower excipient and a higher drug load, the resultant unit dose is smaller which decreases the necessary time for the dissolution or dispersion of the formulation upon oral delivery.

The formulations of the present invention can also comprise further pharmaceutical excipients such as polyvinyl alcohol, polyvinylpyrrolidine, acacia or a combination thereof.

The effect of humidity can have a negative impact on the flowability of particles (e.g., due to cohesiveness). This can be a particular problem with the present invention, which is directed to fast melt multiparticulates which are designed to absorb water. Accordingly, in preferred embodiments, the unit doses of fast melt multiparticulates are premetered prior to actuation of the device. This reduces the contamination of the unit doses as compared to having the formulation in a multiple dose reservoir. Preferably, the premetered unit doses are contained in sachets which minimize the effect of humidity and moisture on the formulation.

Other multiple unit oral dosing devices, adapted contain the formulation in a reservoir or as premetered unit doses, which are useful in the present invention are disclosed in WO01/64182 hereby incorporated by reference.

Classes of drugs which are suitable in the present invention include antacids, anti-inflammatory substances, antibiotics, coronary dilators, cerebral dilators, peripheral vasodilators, anti-infectives, psychotropics, anti-manics, stimulants, anti-histamines, laxatives, decongestants, vitamins, gastro-intestinal sedatives, anti-diarrheal preparations, anti-anginal drugs, vasodilators, anti-arrhythmics, anti-hypertensive drugs, vasoconstrictors and migraine treatments, anti-coagulants and anti-thrombotic drugs, analgesics, anti-pyretics, hypnotics, sedatives, anti-emetics, anti-nauseants, anti-convulsants, neuromuscular drugs, hyper-and hypoglycemic agents, thyroid and anti-thyroid preparations, diuretics, anti-spasmodics, uterine relaxants, mineral and nutritional additives, anti-obesity drugs, anabolic drugs, erythropoietic drugs, anti-asthmatics, bronchodilators, expectorants, cough suppressants, mucolytics, drugs affecting calcification and bone turnover and anti-uricemic drugs.

Specific drugs include gastro-intestinal sedatives such as metoclopramide and propantheline bromide; antacids such as aluminium trisilicate, aluminium hydroxide, ranitidine and cimetidine; anti-inflammatory drugs such as phenylbutazone, indomethacin, naproxen, ibuprofen, flurbiprofen, diclofenac, dexamethasone, prednisone and prednisolone; antibiotics such as clarithromycin, amoxicillin erythromycin, ampicillin, penicillin, cephalosporins, e.g., cephalexin, pharmaceutically acceptable salts thereof and derivatives thereof, coronary vasodilator drugs such as glyceryl trinitrate, isosorbide dinitrate and pentaerythritol tetranitrate; peripheral and cerebral vasodilators such as soloctidilum, vincamine, naftidrofuryl oxalate, co-dergocrine mesylate, cyclandelate, papaverine and nicotinic acid; anti-infective substances such as erythromycin stearate, cephalexin, nalidixic acid, tetracycline hydrochloride, ampicillin, flucloxacillin sodium, examine mandelate and examine hippurate; neuroleptic drugs such as flurazepam, diazepam, temazepam, amitryptyline, doxepin, lithium carbonate, lithium sulfate, chlorpromazine, thioridazine, trifluperazine, fluphenazine, piperothiazine, haloperidol, maprotiline hydrochloride, ipramine, and desmethylimipramine; central nervous stimulants such as methylphenidate, ephedrine, epinephrine, isoproterenol, amphetamine sulfate and amphetamine hydrochloride; antihistamic drugs such as diphenhydramine, diphenylpyraline, chlorpheniramine and brompheniramine; anti-diarrheal drugs such as bisacodyl and magnesium hydroxide; the laxative drug, dioctyl sodium sulfosuccinate; nutritional supplements such as ascorbic acid, alpha tocopherol, thiamine and pyridoxine; anti-spasmodic drugs such as dicyclomine and diphenoxylate; drugs affecting the rhythm of the heart such as verapamil, nifedipine, diltiazem, procainamide, disopyramide, bretylium tosylate, quinidine sulfate and quinidine gluconate; drugs used in the treatment of hypertension such as propranolol hydrochloride, guanethidine monosulphate, methyldopa, oxprenolol hydrochloride, captopril and hydralazine; drugs used in the treatment of migraine such as ergotamine; drugs affecting coagulability of blood such as epsilon aminocaproic acid and protamine sulfate; analgesic drugs such as acetylsalicylic acid, acetaminophen, codeine phosphate, codeine sulfate, oxycodone, dihydrtocodeine tartrate, oxycodeinone, morphine, heroin, nalbuphine, butorphanol tarttate, pentazocine hydrochloride, cyclazacine, pethidine, buprenorphine, scopolamine and mefenamic acid; anti-epileptic drugs such as phenytoin sodium and sodium valproate; neuromuscular drugs such as dantrolene sodium; substances used in the treatment of diabetes such as tolbutamide, disbenase glucagon and insulin; drugs used in the treatment of thyroid gland dysfunction such as triiodothyronine, thyroxine and propylthiouracil, diuretic drugs such as furosemide, chlorthalidone, hydrochlorthiazide, spironolactone and triarnterene; the uterine relaxant drug ritodrine; appetite suppressants such as fenfluramie hydrochloride, phentermine and diethylproprion hydrochloride; anti-asthmatic and bronchodilator drugs such as aminophylline, theophylline, salbutamol, orciprenaline sulphate and terbutaline sulphate; expectorant drugs such as guaiphenesin; cough suppressants such as dextromethorphan and noscapine; mucolytic drugs such as carbocisteine; anti-septics such as cetylpyridinium chloride, tyrothricin and chlorhexidine; decongestant drugs such as phenylpropanolamine and pseudoephedrine; hypnotic drugs such as dichloralphenazone and nitrazepam; anti-nauseant drugs such as promethazine theoclate; haemopoietic drugs such as ferrous sulphate, folic acid and calcium gluconate; uricosuric drugs such as sulphinpyrazone, allopurinol and probenecid; and calcification affecting agents such as biphosphonates, e.g., etidronate, pamidronate, alendronate, residronate, teludronate, clodronate and alendronate.

A particularly preferred active agent is paracetamol (acetaminophen). Other preferred active agents are NSAIDS, such as ibuprofen, indomethacin, aspirin, diclofenac and pharmaceutically acceptable salts thereof.

In certain other embodiments, however, formulations in accordance with the invention do not include any non-steroidal anti-inflammatory drug (SAID).

The size of the unit dose is dependent on the amount of drug needed to provide the intended therapeutic effect and the amount of any pharmaceutically acceptable excipient which may be necessary. Typically, a unit dose of from about 0.01 mg to about 1.5 g would be sufficient to contain a therapeutically effective amount of the drug to be delivered, however, this range is not limiting and can be smaller or higher, depending on the amount of drug and excipient that is necessary.

The following examples serve to illustrate the invention, but should not be understood to be limiting in any respect.

### EXAMPLE 1

Melt coating using xylitol is a preferred embodiment of the invention, as it provides a continuous coating and will therefore be effective in tastemasking. However, the elevated temperatures required to melt-coat xylitol may have deleterious effects on some of the other excipients to be used in the formulation. For example, maltodextrin is clearly useful in fast melt formulations, but charring is observed at higher temperatures.

In this first example, the melt coating process is divided into two stages. Firstly, the xylitol is melt-coated at the required high temperatures. In the second melt coating stage, the remaining excipient materials are added, together with PEG6000. This allows a continuous xylitol coating to be formed without exposing heat-sensitive materials to potentially damaging temperatures. Such a two-stage melt coating process has not been previously disclosed.

The following materials were employed in this example.

| Material | % Composition |
|---|---|
| Paracetamol | 75.55 |
| PEG6000 Powder | 5.00 |
| Xylitol | 12.00 |
| Sodium Starch Glycolate | 2.00 |
| Sodium Bicarbonate | 0.95 |
| Citric Acid Monohydrate | 1.50 |
| Aspartame | 1.50 |
| Acesulphame K | 1.50 |

Method

Granular paracetamol and 12% xylitol were accurately weighed into a glass jar and blended at 42 rpm for 30 minutes using an inversion low shear mixer. The blend was transferred to a jacketed vessel maintained at a temperature of 95° C. The blend was mixed at an impeller speed sufficient to keep the whole powder bed moving (i.e. 222 rpm) using an overhead mixer for a time sufficient to allow homogenous distribution of the molten binder throughout the powder bed. The temperature was then reduced to 60° C. and the PEG6000 powder, sodium starch glycolate, sodium bicarbonate, citric acid monohydrate, aspartame fine and acesulfame potassium added to the blend. The impeller speed was increased to provide continuous movement of the powder bed (i.e. 250 rpm). The formulation was cooled and then sieved using a 710 micron sieve to remove any large agglomerates, once distribution of the melt binder was complete.

Results

The formulation exhibited improved tastemasking compared to Example 2, which is discussed below. This is thought to be due to the formation of a continuous coat of xylitol around the drug crystal. In addition, incorporation of the other excipients at the second melt-coating stage allowed the use of materials which undergo degradation at or near to the melting point of xylitol.

### EXAMPLE 2

10 The following materials were employed in this example.

| Material | % Composition |
|---|---|
| Paracetamol | 73.55 |
| Xylitol | 12.00 |
| PEG6000 Powder | 7.00 |
| Sodium Starch Glycolate | 2.00 |
| Sodium Bicarbonate | 0.95 |
| Citric Acid Monohydrate | 1.50 |
| Acesulphame K | 1.50 |
| Aspartame | 1.50 |

Method

The 1 litre jacketed bowl for a Diosna P1-6 mixer-granulator was heated at 55° C. for 10 minutes before the addition of the granular paracetamol, xylitol, sodium starch glycolate, sodium glycine carbonate, citric acid monohydrate, aspartame fine and acesulphame potassium. This material was blended for a further 10 minutes prior to the addition of the PEG6000. A mixer speed of 50 rpm and a chopper speed of 50 rpm was selected to distribute the binder through the material. Mixing was continued at the elevated temperature for approximately 5 minutes before the bowl was cooled to 25° C. for 10 minutes.

Results

The resulting formulation exhibited poor powder flow and had a tendency to cake upon standing. This poor powder flow is believed to be a consequence of exposed PEG6000 coated surfaces, which are “sticky” in nature. It was found that the addition of any fine material to these formulations was shown to improve flowability, presumably by covering these exposed “sticky” areas. Once these areas have been covered, the continued addition of fine material can eventually lead to a reduction in flowability, as is commonly seen with the addition of fines. The fine material used was 10% Mannitol 35 or talc, and this reduced the cohesive nature of the material and resulted in a formulation with improved powder flowability.

| Formulation | Mannitol 35 | Mannitol | Flodex |
|---|---|---|---|
| Mass (g) | Mass (g) | (% w/w) | Aperture |
| 50.00 | 0.00 | 0.00 | 34+ |
| 50.00 | 1.00 | 1.96 | 34+ |
| 50.00 | 2.50 | 4.76 | 28 |
| 50.00 | 5.00 | 9.09 | 22 |
| 50.00 | 7.50 | 13.04 | 22 |

| Formulation | Talc | Talc | Flodex |
|---|---|---|---|
| Mass (g) | Mass (g) | (% w/w) | Aperture |
| 50.00 | 0.00 | 0.00 | 34+ |
| 50.00 | 1.00 | 1.96 | 32 |
| 50.00 | 1.50 | 4.76 | 30 |
| 50.00 | 2.50 | 9.09 | 26 |
| 50.00 | 5.00 | 13.04 | 18 |
| 50.00 | 7.50 | 13.04 | 16 |

### EXAMPLE 3

The following method details attempts to incorporate spray dried and encapsulated flavouring agents into the formulation

The following materials were employed in this example.

| Order of | | |
|---|---|---|
| Incorporation | Material | % Composition |
| A | Paracetamol | 71.4 |
| A | Erythritol | 10.0 |
| B | PEG6000 Powder | 7.0 |
| A | Sodium Starch Glycolate | 2.0 |
| A | Sodium Glycine Carbonate | 1.2 |
| A | Citric Acid Monohydrate | 1.5 |
| A | Acesulphame K | 1.0 |
| A | Aspartame Fine | 1.0 |
| C | Sweetness Enhancer SD Flavouring | 1.0 |
| D | Sweetness Enhancer Encapsulated | 1.4 |
| | Flavouring | |
| C | Strawberry SD Flavouring | 1.2 |
| D | Vanilla Encapsulated Flavouring | 1.3 |

Method

The 1 litre jacketed bowl for a Diosna P1-6 mixer-granulator was heated at 55° C. for minutes before the addition of all of the materials apart from the PEG6000. This material was blended at the elevated temperature for 10 minutes, to allow thermal equilibration, prior to the addition of the PEG6000. An impeller speed of 50 rpm and a chopper speed of 50 rpm were selected to distribute the binder through the material. Mixing was continued at the elevated temperature for approximately minutes before the bowl was cooled to 25° C. for 15 minutes.

Results

The resulting formulation exhibited pleasant taste, good mouthfeel and a slight bitter aftertaste, which was attributed to the thermal degradation of flavourings. It was found that a better organoleptic profile was achieved if the “A” components were blended and equilibrated at 55° C. for 10 minutes before addition of the “B” component. The formulation was processed at an impeller speed of 50 rpm and a chopper speed of 50 rpm to allow distribution of the binder through the formulation. Mixing was continued at the elevated temperature for approximately 15 minutes before the bowl was cooled to 25° C., at which point the “C” components were added and mixing continued for a further 15 minutes. The “D” components were then added and mixing continued for a further 5 minutes. The addition of the flavours by this method avoids the thermal degradation of the flavouring agents, thereby enhancing the tastemasking properties of the formulations.

The addition of the flavours by this method is preferable to simple dry blending of the materials as incorporation in the melt coat reduces the potential for segregation.

### EXAMPLE 4

The formulations of Examples 1-3 were characterised as having acceptable initial taste, but could have a poor aftertaste attributed to the drug. A formulation was therefore sought which had a good aftertaste. The strategy selected to overcome the poor aftertaste in the formulations in this example, was to reduce the taste of the active agent, so that it is better masked by the flavouring agents. This is done by pre-coating the active agent with an instant release coating. The instant release coating should only delay the drug release whilst the formulation is in the oral cavity, and will preferably not confer grittiness to the formulation.

The following materials were employed in this example.

| Material | % Composition |
|---|---|
| Spray Coated Paracetamol | 73.55 |
| Xylitol | 12.00 |
| PEG6000 Powder | 7.00 |
| Sodium Starch Glycolate | 2.00 |
| Sodium Bicarbonate | 0.95 |
| Citric Acid Monohydrate | 1.50 |
| Acesulphame K | 1.50 |
| Aspartame | 1.50 |

Method
Step 1: Precoating of granular paracetamol

A 15% w/w PVA based aqueous dispersion was prepared and applied to granular paracetamol to a coating level equivalent to a 15% weight gain using a laboratory scale fluid bed drier. The coating module was preheated at 70° C. for 15 minutes with a nominal airflow of 6.0 m³/Hr. The patacetamol was loaded into the coating module and heated to achieve a product temperature of 33-37° C. and the material was fluidised. The dispersion was applied at an atomising pressure of 1.5-2.0 bar. Once the coating had been applied, the pump and atomising air was stopped and the sprayed product was dried. The inlet air temperature was then reduced to 25° C. and the drying operation stopped.

Step 2: Melt Granulation

The 1 litre jacketed bowl for a Diosna P1-6 mixer-granulator was heated at 55° C. for 10 minutes before the addition of the coated paracetamol (prepared in Stage 1), xylitol, sodium starch glycolate, sodium glycine carbonate, citric acid monohydrate, aspartame fine and acesulphame potassium. This material was blended for a further 10 minutes prior to the addition of the PEG6000. An impeller speed of 50 rpm and a chopper speed of 50 rpm were selected to distribute the binder through the material. Mixing was continued at the elevated temperature for approximately 5 minutes before the bowl was cooled to 25° C. for 10 minutes.

Results

It was found that precoating the paracetamol resulted in a formulation with improved organoleptic properties, namely that the formulation exhibited substantially lower aftertaste. Dissolution studies confirmed that there was no apparent difference in the release profile of this material and that of the uncoated paracetamol formulation of Example 2.

### EXAMPLE 5

This example is an extension of Example 4 and investigates the effect of including xylitol in the instant release coating applied to the granular paracetamol. The xylitol is known to act as both as a tastemasking agent and a pore-forming agent and its incorporation in the pre-coating is intended to improve the dispersibility of the formulation whilst retaining the masking of the aftertaste exhibited by the formulation of Example 4.

The following materials were employed in this example.

| Material | % Composition |
|---|---|
| Spray Coated Paracetamol | 73.55 |
| Xylitol | 12.00 |
| PEG6000 Powder | 7.00 |
| Sodium Starch Glycolate | 2.00 |
| Sodium Bicarbonate | 0.95 |
| Citric Acid Monohydrate | 1.50 |
| Acesulphame K | 1.50 |
| Aspartame | 1.50 |

Method
Step 1: Precoating of Granular Paracetamol

A 150% w/w aqueous dispersion was prepared using a proprietary HPMC based polymer system (90% Opadry II High Performance [Colorcon] and 10% xylitol as total solids) applied to granular paracetamol to a coating level equivalent to a 15% weight gain using a laboratory scale fluid bed drier. The coating module was preheated at 70° C. for 15 minutes with a nominal airflow of 6.0 m³/Hr. The paracetamol was loaded into the coating module and heated to achieve a product temperature of 33-37° C. and the material was fluidised. The dispersion was applied at an atomising pressure of 1.5-2.0 bar. Once the coating had been applied, the pump and atomising air was stopped and the sprayed product was dried. The inlet air temperature was then reduced to 25° C. and the drying operation stopped.

Step 2: Melt Granulation

The 1 litre jacketed bowl for a Diosna P1-6 mixer-granulator was heated at 55° C. for 10 minutes before the addition of the coated paracetamol (prepared in Stage 1), xylitol, sodium starch glycolate, sodium glycine carbonate, citric acid monohydrate, aspartame fine and acesulphame potassium. This material was blended for a further 10 minutes prior to the addition of the PEG6000. An impeller speed of 50 rpm and a chopper speed of 50 rpm were selected to distribute the binder through the material. Mixing was continued at the elevated temperature for approximately 5 minutes before the bowl was cooled to 25° C. for 10 minutes.

Results

It was found that precoating the paracetamol resulted in a formulation with improved organoleptic properties, namely that the formulation exhibited substantially lower aftertaste and that the inclusion of xylitol as a pore forming agent (which allows the coating to disperse more rapidly) improved the mouthfeel. Dissolution studies confirmed that there was no apparent difference in the release profile of this material and that of the uncoated paracetamol formulation of Example 2.

### EXAMPLE 6

In this example, an alternative strategy was used to overcome the poor aftertaste in the formulations of Examples 1-3. Instead of reducing the intensity of the aftertaste by pre-coating (as illustrated in Examples 4 & 5), the approach was to prolong the taste intensity of the flavour provided by the flavouring agents. In this example, attempts are made to prolong the flavour profile of the flavouring agents by spray-coating.

The following materials were employed in this example.

| Order of | | |
|---|---|---|
| Incorporation | Material | % Composition |
| A | Paracetamol | 71.4 |
| A | Xylitol | 10.0 |
| B | PEG6000 Powder | 7.0 |
| A | Sodium Starch Glycolate | 2.0 |
| A | Sodium Glycine Carbonate | 1.2 |
| A | Citric Acid Monohydrate | 1.5 |
| A | Acesulphame K | 1.0 |
| A | Aspartame Fine | 1.0 |
| C | Sweetness Enhancer SD Flavouring | 1.0 |
| D | Sweetness Enhancer Encapsulated | 1.4 |
| | Flavouring | |
| C | Strawberry SD Flavouring | 1.2 |
| D | Spray Coated Vanilla Encapsulated | 1.3 |
| | Flavouring | |

Method
Step 1: Spray-Coating of Encapsulated Flavourings

A 15% w/w HPMC-based aqueous dispersion was prepared and applied to the Vanilla Encapsulated flavouring to a coating level equivalent to a 15% weight gain using a laboratory scale fluid bed drier. The coating module was preheated at 70° C. for 15 minutes with a norminal airflow of 6.0 m³/Hr. The Vanilla Encapsulated flavouring was loaded into the coating module and heated to achieve a product temperature of 33-37° C. and the material was fluidised. The dispersion was applied at an atomising pressure of 1.5-2.0 bar. Once the coating had been applied, the pump and atomising air was stopped and the sprayed product was dried. The inlet air temperature was then reduced to 25° C. and the drying operation stopped.

Step 2: Melt Granulation

The 1 litre jacketed bowl for a Diosna P1-6 mixer-granulator was heated at 55° C. for 10 minutes before the addition of the “A” components, which were blended and equilibrated at 55° C. for 10 minutes before the addition of the “B” component. The formulation was processed at an impeller speed of 50 rpm and a chopper speed of 50 rpm to allow distribution of the binder through the material. Mixing was continued at the elevated temperature for approximately 15 minutes before the bowl was cooled to 25° C., at which point the “C” components were added and mixing continued for a further 5 minutes.

Results

This formulation exhibited a longer lasting flavour profile then the formulation of Example 3. As a result, this formulation had a more acceptable aftertaste.

### EXAMPLE 7

This example investigates an alternative method of improving the flowability of the formulation produced in Example 2. Rather than covering the “sticky” areas of exposed PEG6000 by adding fines to the formulation, this example seeks to cover these areas by redistribution of the excipients by prolonged mixing.

The following materials were employed in this example.

| Material | % Composition |
|---|---|
| Paracetamol | 68.55 |
| Xylitol | 12.00 |
| PEG6000 Powder | 7.00 |
| Sodium Starch Glycolate | 2.00 |
| Sodium Bicarbonate | 0.95 |
| Citric Acid Monohydrate | 1.50 |
| Acesulphame K | 1.50 |
| Aspartame | 1.50 |

Method

The 1 litre jacketed bowl for a Diosna P1-6 mixer-granulator was heated at 55° C. for 10 minutes before the addition of the granular paracetamol, xylitol sodium starch glycolate, sodium glycine carbonate, citric acid monohydrate, aspartame fine and acesulphame potassium. This material was blended for a further 10 minutes prior to the addition of the PEG6000. An impeller speed of 50 rpm and a chopper speed of 50 rpm were selected to distribute the binder through the material. Mixing was continued at the elevated temperature for approximately 5 minutes before the bowl was cooled to 25° C. for 40 minutes.

Results

It was found that, by extending the cooling period from 10 to 40 minutes, the tendency of the formulation to cake upon standing was greatly reduced and the resultant formulation was free-flowing.

### EXAMPLE 8

This example investigates the effect of the particle size ranges of the materials used to make the fast melt formulations.

The following materials are employed in this example.

| Material | % Composition |
|---|---|
| Chlorpheniramine Maleate | 8.0 |
| Mannitol | 69.3 |
| Xylitol | 15.0 |
| Sodium Starch Glycolate | 2.0 |
| Sodium Glycine Carbonate | 1.2 |
| Citric Acid Monohydrate | 1.5 |
| Acesulphame K | 1.5 |
| Aspartame Fine | 1.5 |

Method

The mannitol particles used have a particle size range of 70 to 125 μm and the other components are approximately 10% of the particle size of the mannitol. The materials are accurately weight into a beaker. The material is then transferred to a Hosokawa AMS-MINI, equipped with a 5 mm gap rotor, via a funnel attached to the largest port in the lid with the equipment running at 3.5% of the maximum speed. The port is sealed and the cooling water switched on. The equipment is then run at 20% maximum speed for 5 minutes, followed by 50% maximum speed for 10 minutes. The equipment is then switched off, dismantled and the resulting formulation is recovered mechanically.

Results

The benefits over previous formulations are expected to be: (a) reduced excipient payload and (b) more efficient coating/embedding of the core particle with the drug/excipient. Mechanofusion also allows equivalent formulations to be prepared at lower temperatures than are possible using the technique of melt granulation.

1. A pharmaceutical formulation comprising a free-flowing plurality of particles comprising a pharmaceutically active agent and an excipient, wherein the formulation includes one or more tastemasking agents incorporated into the formulation so that the taste intensity of the tastemasking agents substantially always exceeds the taste intensity of the active agent, without significantly affecting the dissolution profile of the formulation.

2. A pharmaceutical formulation as claimed in claim 1, wherein said particles each include both the active agent and the excipient.

3. A pharmaceutical formulation as claimed in claim 2, wherein the particles comprise a core and a coating that includes a quantity of the excipient.

4. A pharmaceutical formulation as claimed in claim 3, wherein the coating is a continuous coating, surrounding the core.

5. A pharmaceutical formulation as claimed in claim 1, wherein the particles are formed by melt-coating core particles with a coating material that includes a quantity of the excipient, at a temperature below the melting point or decomposition temperature of the active agent.

6. A pharmaceutical formulation as claimed in claim 5, wherein the core particles are 10 to 1000 μm in size.

7. A drug formulation as claimed in claim 5, wherein the size of the excipient particles used to melt-coat the core particles is 10% or less than the size of the core particle.

8. A pharmaceutical formulation as claimed in claim 3, wherein a quantity of the active agent is included in the core.

9. A pharmaceutical formulation as claimed in claim 1, wherein the formulation includes one or more sweeteners and/or flavouring agents.

10. A pharmaceutical formulation as claimed in claim 3, wherein a quantity of the tastemasking agents is included in the coating.

11. A pharmaceutical formulation as claimed in claim 3, wherein the core or is not pre-coated with a release retarding coating.

12. A pharmaceutical formulation as claimed in claim 3, wherein the coating further comprises a water soluble or hydrophilic binder.

13. A pharmaceutical formulation as claimed in claim 3, wherein the coating further comprises a hydrophobic binder.

14. A pharmaceutical formulation as claimed in claim 12, wherein the binder melts or softens sufficiently to melt-coat the core particles at a temperature below the melting point or decomposition temperature of the active agent.

15. A pharmaceutical formulation as claimed in claim 3, wherein the excipient melts or softens sufficiently to melt-coat the core particles at a temperature below the melting point or decomposition temperature of the active agent.

16. A pharmaceutical formulation as claimed in claim 14, wherein the binder melts or softens sufficiently to melt-coat the core particles at a temperature below the melting point or decomposition temperature of the excipient.

17. A pharmaceutical formulation as claimed in claim 3, wherein the core or core particles include a water soluble excipient.

18. A pharmaceutical formulation as claimed in claim 1, formed by a process in which the active agent is not raised to or above its melting point, or a temperature at which a significant proportion thereof is caused to decompose.

19. A pharmaceutical formulation as claimed in claim 17, wherein the water soluble excipient is one or more of: sugars, sugar alcohols, polyethylene glycols (PEGs), polyethylene oxides, gelatin, partially hydrolyzed gelatin, hydrolyzed dextran, dextrin, alginate, sodium bicarbonate, citric acid, tartaric acid, malic acid, fumaric acid, adipic acid, succinic acid, sodium glycine carbonate and sweeteners.

20. A pharmaceutical formulation as claimed in claim 19, wherein the water soluble excipient is a sugar alcohol or combination of sugar alcohols.

21. A pharmaceutical formulation as claimed in claim 20, wherein the sugar alcohol is selected from the group consisting of sorbitol, mannitol, maltitol, reduced starch saccharide, xylitol, reduced paratinose, erythritol, and combinations thereof.

22. A pharmaceutical formulation as claimed in claim 12, wherein the binder includes one or more of: polyethylene glycols (PEGs), polyethylene oxides, sugar alcohols, stearic acid, glyceryl monostearate, glyceryl palmitostearate and suppository bases.

23. A pharmaceutical formulation as claimed in claim 3, wherein the core includes an additional excipient for controlling or delaying the release of the active agent.

24. A pharmaceutical formulation as claimed in claim 23, wherein the core includes a layer or coating of said additional excipient encapsulating an inner core comprising the active agent.

25. A pharmaceutical formulation as claimed in claim 23, wherein said additional excipient provides an enteric or sustained release coating.

26. A pharmaceutical formulation as claimed in claim 25, wherein said additional excipient is selected from the group consisting of cellulose acetate phthalate, hydroxypropylmethylcellulose phthalate, polymethacrylates, shellac, ethylcellulose, hydroxypropylcellulose, and hydroxypropylmethylcellulose.

27. A pharmaceutical formulation as claimed in claim 1, wherein said formulation dissolves in a patient's mouth within 30 or 15 seconds after administration without the coadministration of a fluid.

28. A pharmaceutical formulation as claimed in claim 1, wherein the particles comprise at least about 50% active agent.

29. A pharmaceutical formulation as claimed in claim 1, wherein the particles comprise less than about 50% active agent.

30. A pharmaceutical formulation as claimed in claim 1 further comprising a low viscosity polymer.

31. A pharmaceutical formulation as claimed in claim 1 further comprising a salivary stimulant.

32. A pharmaceutical formulation as claimed in claim 1, wherein said formulation further comprises an excipient selected from the group consisting of polyvinyl alcohol, polyvinylpyrrolidine, acacia and combinations thereof.

33. A pharmaceutical formulation as claimed in claim 1 further comprising a water soluble artificial sweetener.

34. A pharmaceutical formulation as claimed in claim 33, wherein said water soluble artificial sweetener is selected from the group consisting of soluble saccharin salts, such as sodium or calcium saccharin salts, cyclamate salts, acesulfam-K, the free acid form of saccharin and mixtures thereof.

35. A pharmaceutical formulation as claimed in claim 1 further comprising a dipeptide based sweetener.

36. A pharmaceutical formulation as claimed in claim 35, wherein said dipeptide based sweetener is L-aspartyl L-phenylalanine methyl ester.

37. A pharmaceutical formulation as claimed in claim 31, wherein said salivary stimulant is selected from the group consisting of citric acid, tartaric acid, malic acid, fumaric acid, adipic acid, succinic acid, acid anhydrides thereof, acid salts thereof and combinations thereof.

38. A pharmaceutical formulation as claimed in claim 31, wherein said salivary stimulant is an effervescent agent.

39. A pharmaceutical formulation as claimed in claim 38, wherein said effervescent agent is the result of a reaction of a soluble acid source and an alkali metal carbonate or carbonate source.

40. A pharmaceutical formulation as claimed in claim 1, wherein the formulation is capable of dissolving or dispersing in a patient's mouth within 1 minute after administration without the co-administration of a fluid.

41. A pharmaceutical formulation as claimed in claim 1, arranged for direct un-encapsulated administration to the oral cavity.

42. A pharmaceutical formulation as claimed in claim 1, wherein the particles are non-compressed.

43. A pharmaceutical formulation as claimed in claim 1, wherein a flavouring intensity substantially always exceeds the intensity of the taste of the active agent, without affecting the dissolution profile of the formulation.

44. A method of preparing a formulation as claimed in claim 1, comprising forming the particles by melt-coating core particles with a coating material that includes a quantity of the water-soluble excipient and, optionally, a quantity of the binder, at a temperature below the melting point or decomposition temperature of the active agent.

45. A method of treating a human or animal patient, the method comprising preparing the drug formulation of claim 1 and administering the formulation directly in an un-encapsulated form to the patient's oral cavity.

46. The method of claim 45, wherein a drug formulation prepared by a method as claimed in claim 44.

47. A drug delivery system comprising a dosing device comprising a housing and an actuator, said device containing at least one unit dose of a drug formulation as claimed in claim 1, said device upon actuation delivering a unit dose of said drug formulation such that an effective dose of said drug cannot be delivered into the lower lung of a human patient.

48. The drug delivery system as claimed in claim 47, wherein said at least one unit dose is contained in a reservoir.

49. The drug delivery system as claimed in claim 47, further comprising a metering component to meter a unit dose from said reservoir upon actuation of said system.

50. The drug delivery system as claimed in claim 47, comprising multiple unit doses, wherein said unit doses are individually metered prior to said actuation.

51. The drug delivery system as claimed in claim 47, further comprising sachets, each sachet containing said individually metered unit dose.

52. A method of treating a patient comprising administering a formulation as claimed in claim 1 for gastrointestinal deposition.

53. A method as claimed in claim 44, further comprising melt granulating said water soluble excipient and the active agent to form a homogenous mixture.

54. A method as claimed in claim 44, further comprising melt coating said water soluble excipient onto said active agent.

55. A method as claimed in claim 44, which are prepared without the use of an aqueous fluid.

56. A pharmaceutical formulation as claimed in claim 6, wherein the core particles are 200 to 600 μm in size.

57. A pharmaceutical formulation as claimed in claim 6, wherein the core particles are 100 to 300 μm in size.

58. A pharmaceutical formulation as claimed in claim 13, wherein the binder melts or softens sufficiently to melt-coat the core particles at a temperature below the melting point or decomposition temperature of the active agent.

59. A pharmaceutical formulation as claimed in claim 1, wherein the particles comprise at least about 60% active agent.

60. A pharmaceutical formulation as claimed in claim 1, wherein the particles comprise at least about 75% active agent.
